(19) United States
(12) Patent Application Publication
(10) Pub. No.: US 2026/0142026 A1
(43) Pub. Date: May 21, 2026

(54) ARCHITECTURE FOR INTEROPERABLE UROLOGY OPERATING ROOM

(71) Applicant: Boston Scientific Scimed, Inc., maple grove, MN (US)

(72) Inventors: Jeffrey A. Meganck, N Grafton, MA (US); Niraj Prasad Rauniyar, Plymouth, MN (US); Hernan Altman, Tivon (IL); Christopher Stankus, Maple Grove, MN (US)

(73) Assignee: Boston Scientific Scimed, Inc., Maple Grove, MN (US)

(21) Appl. No.: 19/357,681

(22) Filed: Oct. 14, 2025

Related U.S. Application Data
(60) Provisional application No. 63/707,003, filed on Oct. 14, 2024.

(51) Int. Cl. G16H 40/63; G16H 15/00

(52) U.S. Cl. CPC G16H 40/63; G16H 15/00

(57) ABSTRACT

The disclosure provides a centralized operating room (OR) controller and associated components for an interoperable OR. The interoperable OR can be provisioned for a urological procedure. The centralized controller provides for interaction with each device in the OR and provides for configuration, monitoring, and control of the devices in a composite group or groups.

### CROSS REFERENCE TO RELATED APPLICATIONS

This application claims the benefit of priority under 35 U.S.C. § 119 to U.S. Provisional Patent Application No. 63/707,003, filed Oct. 14, 2024, each of which is herein incorporated by reference in its entirety.

### TECHNICAL FIELD

The disclosure generally relates to urology operating rooms and particularly to interoperability of the components within a urology operating room.

### BACKGROUND

A modern urological operating room (OR) includes several complex devices each employing different technologies and systems. These several different devices are used together to perform urological procedures on a patient. The ability to leverage all these devices increases as the complexity and number of devices used to perform urological procedures increases. This ever-increasing complexity results in an increase in both the cognitive and physical burdens to the physicians, nurses, and assistants carrying out the procedure. For example, each piece of equipment used in the OR needs to be configured prior to the procedure and then monitored and controlled during the procedure. Adding to this burden, each piece of equipment typically has its own custom computing hardware configuration and display.

The various technologies with which each device in the OR relies on as well as the various computing hardware specification and display results in the need to monitor and control each device individually. As the number of devices provisioned in a urological OR increases, it becomes an untenable burden for the configure, monitor, and control each of them for the modern physician.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The disclosure provides components for an interoperable urology operating room (OR) including a centralized controller configured to interact with each device in the OR to provide for configuration, monitoring, and control of the devices in a composite group or groups.

In some embodiments, the disclosure can be implemented as a method for a centralized operating theater controller. The method can comprise receiving, from processing circuitry of at least one of a plurality of therapy consoles, an indication of physiological information related to a urological procedure; generating a plurality of graphical elements comprising visual indications of the physiological information; generating a composite display based on the plurality of graphical elements; and sending control signals to an operating theater display to cause the operating theater display to display the composite display, wherein the operating theater display is provisioned in an operating theater of a urological operating room (OR) suite, and wherein the plurality of therapy consoles are provisioned in the operating theater and configured to be utilized simultaneously to perform the urological procedure in the urological OR suite.

With further embodiments, the method can comprise determining other physiological information from the received physiological information, wherein the plurality of graphical elements further comprises visual indications of the other physiological information.

With further embodiments of the method, the plurality of therapy consoles comprises at least a fluidics unit, a laser energy console, and an endoscope, and the received physiological information comprises one or more of a pressure, a fluid flow, an energy, or a power, and wherein the determined other physiological information comprises a turbidity, a clarity, a temperature, a procedure state, or a target tissue state.

With further embodiments, the method can comprise receiving, from the processing circuitry of at least one of the plurality of therapy consoles, an indication of captured scene information, wherein the plurality of graphical elements further comprises visual indications of the captured scene information.

With further embodiments of the method, the composite display is a first composite display, and the method can further comprise generating a second composite display based on one or more first ones of the plurality of graphical elements; and sending control signals to an observation room display to cause the observation room display to display the second composite display, wherein the first composite display is generated based on one or more second ones of the plurality of graphical elements, and wherein the observation room display is provisioned in an observation room of the urological OR suite.

With further embodiments of the method, the one or more first ones of the plurality of graphical elements are different than the one or more second ones of the plurality of graphical elements and wherein the first composite display is different than the second composite display.

With further embodiments, the method can comprise receiving, from at least one of one or more equipment controls, at least one control signal, the at least one control signal comprising indications to cause a one of the plurality of therapy consoles to change a parameter or maintain a parameter; and sending the at least one control signal to the one of the plurality of therapy consoles.

With further embodiments, the method can comprise processing the at least one control signal, wherein the processed at least one control signal is sent to the one of the plurality of therapy consoles.

With further embodiments, the method can comprise sending the processed at least one control signal to another one of the plurality of therapy consoles.

With further embodiments, the method can comprise receiving, from the one of the plurality of therapy consoles, a feedback signal; processing the received feedback signal; and sending the processed feedback signal to the at least one of the one or more equipment controls, wherein the processed feedback signal causes the at least one of the one or more equipment controls to provide feedback to a user.

With further embodiments of the method, the one or more equipment controls are provisioned in the operating theater.

With further embodiments, the method can comprise generating configuration signals for the plurality of therapy consoles based in part on procedure preferences for the urological procedure; and sending the configuration signals to the plurality of therapy consoles.

In some embodiments, the disclosure can be implemented as a urological operating room suite. The urological operating room suite can comprise an operating theater; a plurality of therapy consoles provisioned in the operating theater, each of the plurality of therapy consoles comprising processing circuitry, wherein the plurality of therapy consoles are configured to be utilized simultaneously to perform a urological procedure in the operating room (OR) suite; an operating theater display provisioned in the operating theater; and a centralized operating theater controller, the centralized operating theater controller comprising a processor, and a memory storage device coupled to the processor, the memory storage device comprising instructions that when executed by the processor cause the centralized operating theater controller to implement any of the methods described herein.

In some embodiments, the disclosure can be implemented as at least one machine readable storage device. The storage device can comprise a plurality of instructions that in response to being executed by a processor of a centralized operating theater controller cause the centralized operating theater controller to implement the any of the methods described herein.

In some embodiments, the disclosure can be implemented as a urological operating room suite. The urological operating room suite can comprise an operating theater; a plurality of therapy consoles provisioned in the operating theater, each of the plurality of therapy consoles comprising processing circuitry, wherein the plurality of therapy consoles are configured to be utilized simultaneously to perform a urological procedure in the operating room (OR) suite; an operating theater display provisioned in the operating theater; and a centralized operating theater controller, the centralized operating theater controller comprising a processor and a memory storage device coupled to the processor. The memory storage device can comprise instructions that when executed by the processor cause the centralized operating theater controller to receive, from the processing circuitry of at least one of the plurality of therapy consoles, an indication of physiological information related to the urological procedure; generate a plurality of graphical elements comprising visual indications of the physiological information; generate a composite display based on the plurality of graphical elements; and send control signals to the operating theater display to cause the operating theater display to display the composite display.

With further embodiments of the urological operating room suite, the instructions when executed by the processor, further cause the centralized operating theater controller to determine other physiological information from the received physiological information, wherein the plurality of graphical elements further comprises visual indications of the other physiological information.

With further embodiments of the urological operating room suite, the plurality of therapy consoles comprises at least a fluidics unit, a laser energy console, and an endoscope, and the received physiological information comprises one or more of a pressure, a fluid flow, an energy, or a power, and wherein the determined other physiological information comprises a turbidity, a clarity, a temperature, a procedure state, or a target tissue state.

With further embodiments of the urological operating room suite, the instructions when executed by the processor, further cause the centralized operating theater controller to receive, from the processing circuitry of at least one of the plurality of therapy consoles, an indication of captured scene information, wherein the plurality of graphical elements further comprises visual indications of the captured scene information.

With further embodiments, the urological operating room suite can comprise an observation room and an observation room display provisioned in the observation room, wherein the composite display is a first composite display and wherein the instructions when executed by the processor, further cause the centralized operating theater controller to generate a second composite display based on one or more first ones of the plurality of graphical elements; and send control signals to the observation room display to cause the observation room display to display the second composite display, wherein the first composite display is generated based on one or more second ones of the plurality of graphical elements.

With further embodiments of the urological operating room suite, the one or more first ones of the plurality of graphical elements are different than the one or more second ones of the plurality of graphical elements and wherein the first composite display is different than the second composite display.

With further embodiments, the urological operating room suite can comprise one or more equipment controls, wherein the instructions when executed by the processor, further cause the centralized operating theater controller to receive, from at least one of the one or more equipment controls, at least one control signal, the at least one control signal comprising indications to cause a one of the plurality of therapy consoles to change a parameter or maintain a parameter; and send, to the one of the plurality of therapy consoles, the at least one control signal.

With further embodiments of the urological operating room suite, the instructions when executed by the processor, further cause the centralized operating theater controller to process the at least one control signal, wherein the processed at least one control signal is sent to the one of the plurality of therapy consoles.

With further embodiments of the urological operating room suite, the instructions when executed by the processor, further cause the centralized operating theater controller to send the processed at least one control signal to another one of the plurality of therapy consoles.

With further embodiments of the urological operating room suite, the instructions when executed by the processor, further cause the centralized operating theater controller to receive, from the one of the plurality of therapy consoles, a feedback signal; process the received feedback signal; and send, to the at least one of the one or more equipment controls, the processed feedback signal, wherein the processed feedback signal causes the at least one of the one or more equipment controls to provide feedback to a user.

With further embodiments of the urological operating room suite, the one or more equipment controls are provisioned in the operating theater.

With further embodiments of the urological operating room suite, the instructions when executed by the processor, further cause the centralized operating theater controller to generate configuration signals for the plurality of therapy consoles based in part on procedure preferences for the urological procedure; send, to the plurality of therapy consoles, the configuration signals.

In some embodiments, the disclosure can be implemented as at least one machine readable storage device. The storage device can comprise a plurality of instructions that in response to being executed by a processor of a centralized operating theater controller cause the centralized operating theater controller to receive, from processing circuitry of at least one of a plurality of therapy consoles, an indication of physiological information related to a urological procedure; generate a plurality of graphical elements comprising visual indications of the physiological information; generate a composite display based on the plurality of graphical elements; and send control signals to an operating theater display to cause the operating theater display to display the composite display, wherein the operating theater display is provisioned in an operating theater of a urological operating room (OR) suite, and wherein the plurality of therapy consoles are provisioned in the operating theater and configured to be utilized simultaneously to perform the urological procedure in the urological OR suite.

With further embodiments of the at least one machine readable storage device, the instructions when executed by the processor, further cause the centralized operating theater controller to receive, from at least one of one or more equipment controls, at least one control signal, the at least one control signal comprising indications to cause a one of the plurality of therapy consoles to change a parameter or maintain a parameter; and send, to the one of the plurality of therapy consoles, the at least one control signal.

With further embodiments of the at least one machine readable storage device, the instructions when executed by the processor, further cause the centralized operating theater controller to receive, from the one of the plurality of therapy consoles, a feedback signal; process the received feedback signal; and send, to the at least one of the one or more equipment controls, the processed feedback signal, wherein the processed feedback signal causes the at least one of the one or more equipment controls to provide feedback to a user.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates a first operating room (OR) environment in accordance with at least one embodiment.

FIG. 1B illustrates a centralized operating theater controller of the first OR environment of FIG. 1A in greater detail.

FIG. 2A, FIG. 2B, FIG. 2C, FIG. 2D, FIG. 2E, and FIG. 2F illustrate a second OR environment, provisioned for a urological procedure, in accordance with at least one embodiment.

FIG. 3A illustrates a third OR environment in accordance with at least one embodiment.

FIG. 3B illustrates a centralized operating theater controller of the third OR environment of FIG. 3A in greater detail.

FIG. 4A illustrates a first logic flow in accordance with at least one embodiment.

FIG. 4B illustrates a second logic flow in accordance with at least one embodiment.

FIG. 5 illustrates a centralized operating theater controller in accordance with at least one embodiment.

FIG. 6 illustrates a third logic flow in accordance with at least one embodiment.

FIG. 7 illustrates a fourth logic flow in accordance with at least one embodiment.

FIG. 8 illustrates a computer-readable storage medium in accordance with at least one embodiment.

### DETAILED DESCRIPTION

The present disclosure is described with reference to medical devices, methods, and systems. Often, the disclosure is described with reference to surgical urological equipment and procedures. For example, in some procedures, a medical device (e.g., an endoscope, a laser fiber, a snare, a basket, etc.) may be advanced through a path or passage in a body (e.g., a ureter) to aid in removal of target tissue (e.g., a stone, or the like) from a cavity in the body (e.g., a calyx of a kidney). In another example, a medical device (e.g., an endoscope, a laser fiber, a morcellator, etc.) may be advanced through a path or passage in a body (e.g., a ureter) to aid in treatment and/or removal of target tissue (e.g., cancerous prostate tissue, or the like).

It is to be appreciated that references to a particular type of procedure, medical device, target tissue, or body passage or cavity are provided for convenience and clarity of describing the invention and are not intended to limit the claims beyond what is specified in each claim.

The terms “proximal” and “distal” may be utilized along with terms such as “parallel,” “transverse,” and “longitudinal” to describe the relative relationship and position of elements described herein. Proximal refers to a position closer to the exterior of the body (or closer to a user), whereas distal refers to a position closer to the interior of the body (or further away from the user). Further, the term “elongated” is often used herein refers to an object that is substantially longer in one direction (e.g., referred to as the longitudinal direction) in relation to a perpendicular direction. For example, an object having a longer width than length could be referred to herein as elongated.

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its structure and method, together with further objects and advantages will be better understood from the following description when read in conjunction with the accompanying figures.

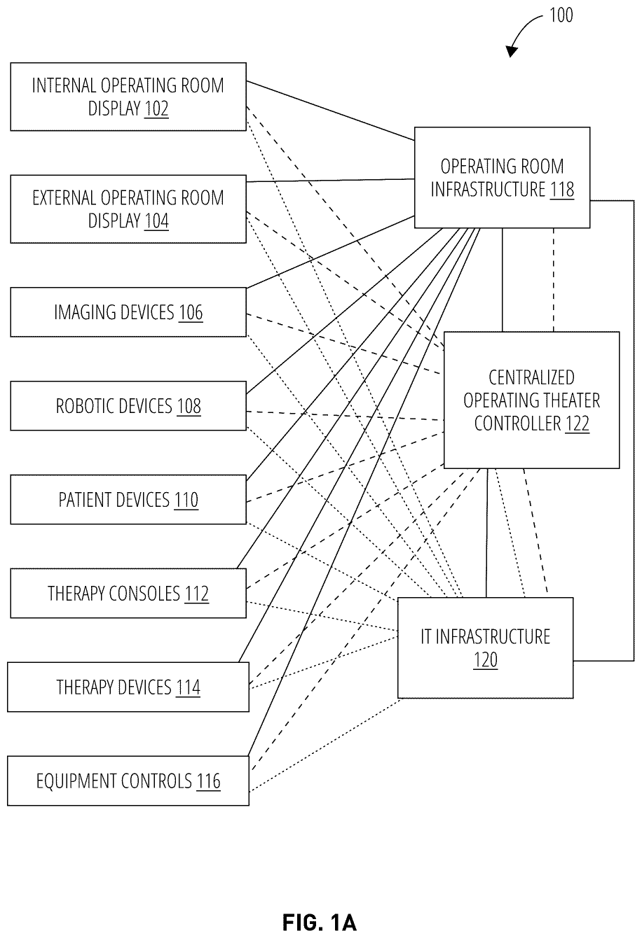

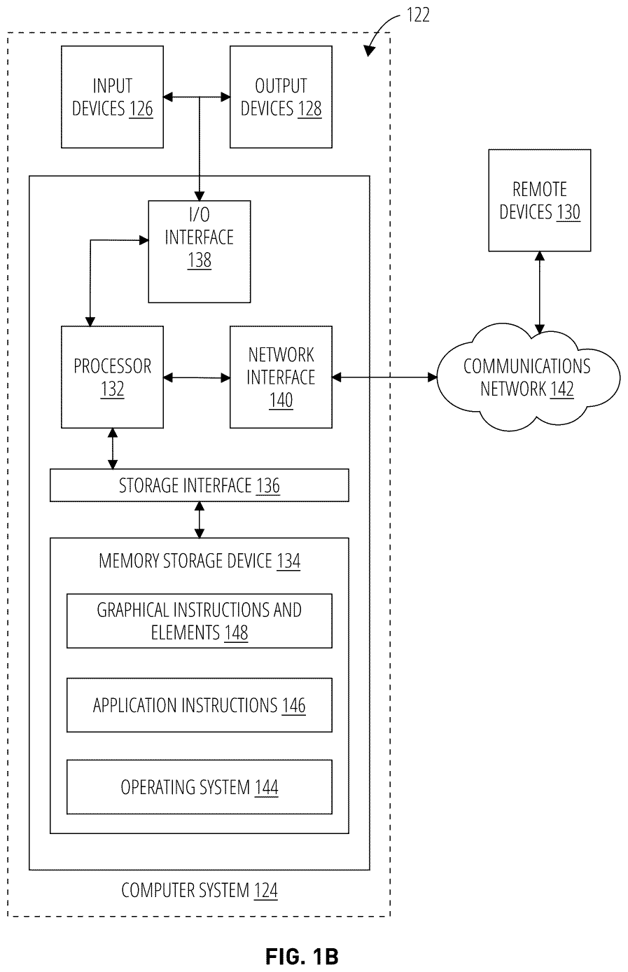

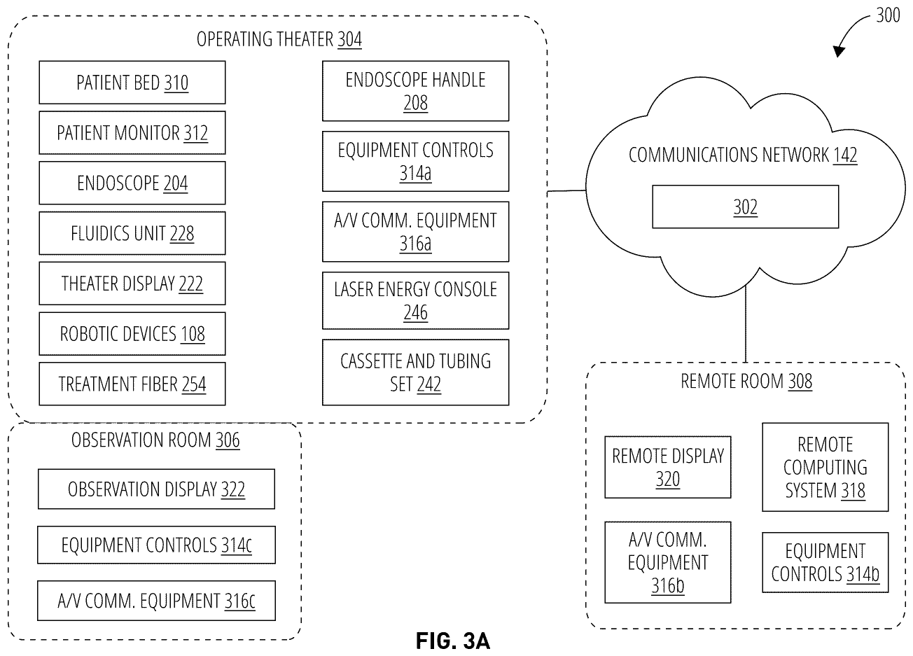

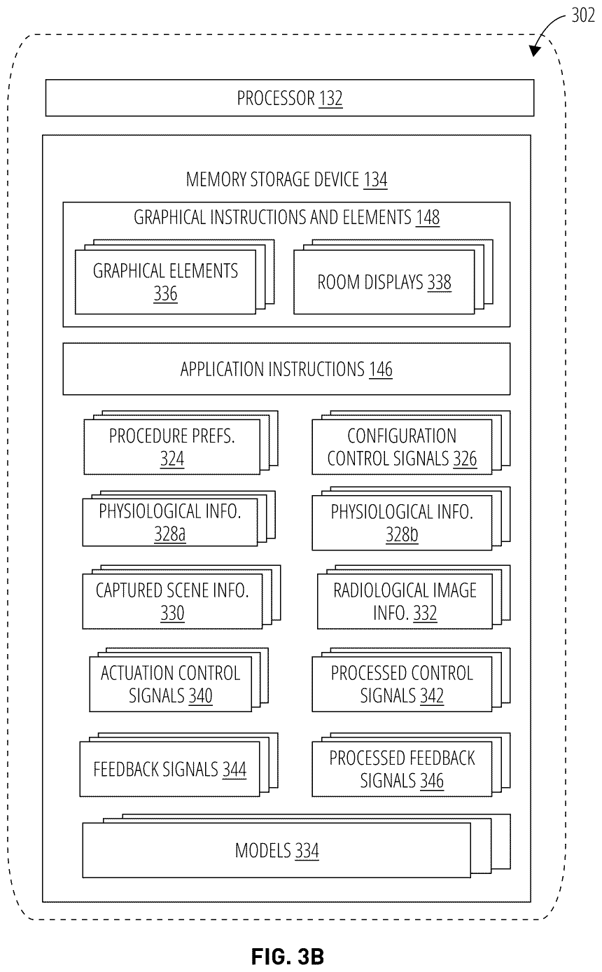

Numerous aspects of the present disclosure are now described with reference to an illustrative operating room (OR) environment 100 as depicted in FIG. 1A. The OR environment 100 will often be focused on an operating theater (or surgical suite), which is a designated room or space where the patient and procedure take place. However, the OR environment 100, and particularly, the equipment of OR environment 100 can be disposed and/or used in multiple locations including the operating theater (e.g., see FIG. 3A). Locations outside or other than the operating theater can include a viewing room, a control room, a computing infrastructure room, a proctor viewing room, a remote physician operating room, or the like). As will be appreciated, not all locations in which equipment of OR environment 100 will be deployed are sterile. Further, even in the operating theater itself there is often a delineation between a sterile and non-sterile field.

OR environment 100 can include internal and external displays. For example, OR environment 100 can include an internal operating room display 102 (or displays) arranged to display information within the operating theater. Similarly, OR environment 100 can include external operating room display 104 (or displays) arranged to display information outside the operating theater, for example, to observers, to remote physicians, to proctors, to nurses or admins outside the sterile field or in another room. Further, OR environment 100 can include imaging devices 106, robotic devices 108, patient devices 110, therapy consoles 112, therapy devices 114, equipment controls 116, operating room infrastructure 118, information technology (IT) infrastructure 120, and centralized operating theater controller 122.

With some embodiments, internal operating room display 102 can include an overhead operating theater display. As a specific example, internal operating room display 102 can include a display physically located inside of the operating theater (e.g., a wall mounted monitor, a ceiling mounted monitor, a monitor mounted on an articulating arm, or the like) that is visible to multiple observers. In some embodiments, internal operating room display 102 can include a wearable display, such as, a heads-up display, a virtual reality display, an augmented reality display, a tablet or small form factor display and associated harness to wear the display. With some embodiments, multiple wearable displays can be provided. For example, the physician can wear a head mounted display while an assistance wears a tablet computer. In some embodiments, internal operating room display 102 can include an on-patient display, for example, a display or monitor physical attached to the patient, a display projected onto the patient, or the like.

In some embodiments, external operating room display 104 can include a monitor or monitors located outside the operating theater and configured to display information from inside the operating theater to persons outside the operating theater. For example, external operating room display 104 can include a monitor or monitors configured to display a view (or views) of the surgical suite. In some examples, the external operating room display 104 can be located proximate to (e.g., in the next room, or the like) the surgical suite while in other examples, the external operating room display 104 is in another area of the premises, off premises (e.g., in another geographical location, or the like). In some embodiments the external operating room display 104 can be configured for direct real-time viewing, recorded viewing, or both. With some embodiments, external operating room display 104 can include a monitor or monitors configured to display information from the surgical suite or components of the OR environment 100. For example, external operating room display 104 could be a monitor configured to mirror the displayed contents on a monitor associated with another piece of equipment in the surgical suite (e.g., vital monitoring equipment, therapy console, or the like. It is noted that equipment provisioned in the OR environment 100 can have its own display. Examples of these displays are described herein and can be classified as internal operating room display 102 or external operating room display 104 depending upon the location of the equipment.

Imaging devices 106 can include any of a variety of imaging devices configured to capture images of the patient, either pre-procedure, intra-procedure, or post-procedure. The imaging devices 106 can utilize any of several imaging modalities (e.g., radiography, ultrasound, tomographic, direct visualization, or the like). With some embodiments, imaging devices 106 can include a planar X-ray device, a fluoroscopy device, or the like. In some embodiments, imaging devices 106 can include an ultrasound imaging device. In some embodiments, imaging devices 106 can include a magnetic resonance imaging (MRI) device, a computed tomography (CT) scanning device, a positron emission tomography (PET)-MRI device, single-photon emission (SPE)-CT scanning device, or the like.

Robotic devices 108 can include any of a variety of robotic equipment configured to automatically or under control of a user, observe and/or assist in the procedure. For example, the robotic devices 108 can be equipment configured to provide a surgical navigation system (e.g., device, motion, body part tracking, or the like) and computing resources (e.g., processing circuitry, memory, etc.) configured to provide real-time tracking (e.g., needle tracking, therapy device tracking, or the like) or something in the operating theater (e.g., a body part, a medical device, or the like). In some examples, robotic devices 108 can include motion control, articulation, grasping to facilitate automatic movement, analysis, or control of equipment in the OR environment 100. Additionally, robotic devices can be configured to manipulate ones of the devices (e.g., therapy devices 114, or the like) automatically and/or under remote or non-contact control from a physician.

Patient devices 110 can include any equipment in direct contact with the patient, for example, anesthesia equipment, vital monitoring equipment, the surgical bed, and surgical bed accessories. With some embodiments, patient devices 110 can include equipment to provide general or local anesthesia to the patient, such as, a continuous-flow anesthetic machine, or the like. As another example, patient devices 110 can include continuous bedside monitors (e.g., for temperature, pulse, etc.), hemodynamic monitors, respiratory monitors, neurological monitors, cardiac monitors, or the like.

Therapy consoles 112 and therapy devices 114 can comprise any equipment (e.g., capital equipment, single use devices, reusable devices, or the like) arranged to perform or provide the treatment associated with the procedure. In general, therapy consoles 112 can include any capital equipment and/or infrastructure used for delivery of the desired treatment or therapy. For example, 112 can include visualization equipment, such as, endoscope viewing and/or control consoles. As another example, therapy consoles 112 can include fluidic consoles to provide fluid inflow and/or outflow, suction, or the like. In another example, therapy consoles 112 can include lithotripsy equipment, such as, laser consoles configured to generate laser energy to ablate, fragment, dust, or otherwise treat calculi. With another example, therapy consoles 112 can include soft tissue therapy equipment, such as, morcellation consoles, ablation consoles, resection consoles, biopsy consoles, cauterization consoles (e.g., laser, electrocautery, radio frequency (RF) cautery, or the like).

Therapy devices 114 can include any device used with the therapy consoles 112 to affect the treatment or procedure. For example, therapy devices 114 can include endoscopes, such as, a ureteroscope, a cystoscope, a nephroscope, or a resectoscope. The endoscopes can be electronic or optical and can be configured to visualize the anatomy in minimally invasive procedures. With some examples, therapy devices 114 can include retrieval devices (e.g., baskets, snares, loops, hooks, pinchers, or the like). In some examples, therapy devices 114 can include optical fibers to convey laser energy to a treatment site, morcellation devices, energy delivery devices (e.g., thermal, electric, RF, or the like). In some examples, therapy devices 114 can include post-procedure or healing devices, such as, stents and stent delivery devices, or the like. Any of the therapy devices 114 can be single use devices, reusable devices, or therapy devices 114 can include a combination of single use and reusable devices.

Equipment controls 116 includes all equipment and/or interfaces used to control equipment in OR environment 100 and/or facilitate exchange of data between equipment in OR environment 100. Examples of such controls are provided throughout this disclosure.

Operating room infrastructure 118 can include any equipment built into the physical infrastructure of the surgical suite. For example, operating room infrastructure 118 can include operating theater lighting (e.g., wall mounted, ceiling mounted, mounted to an articulating arm, or the like) configured to provide illumination of the room and/or surgical field. In some examples, operating room infrastructure 118 can include image and/or audio capture devices (e.g., video cameras, or the like). In some examples, operating room infrastructure 118 can include centralized air, water, and/or gas supply lines (e.g., suction, filtered water, oxygen, etc.) In some examples, operating room infrastructure 118 can include central waste collection systems (e.g., floor drain, or the like). In some examples, operating room infrastructure 118 can include warming systems (e.g., warming oven, or the like) configured to warm consumables used during a procedure. With some examples, operating room infrastructure 118 can include electrical power supplies and can include hardwired or mobile power supplies.

IT infrastructure 120 can include any structure and equipment used for the transmission, storage, and/or processing of data used in the procedure. For example, IT infrastructure 120 can include servers, data storage arrays, data centers, wire and/or wireless communication cabling and equipment, medical record data storage devices, or the like.

As depicted, the components of OR environment 100 are coupled to centralized operating room infrastructure 118 and IT infrastructure 120. For example, internal operating room display 102, external operating room display 104, imaging devices 106, robotic devices 108, patient devices 110, therapy consoles 112, and/or therapy devices 114 can be configured to receive and/or transmit data (e.g., information elements, control signals, etc.) via IT infrastructure 120. Such exchange of data can be unidirectional or bidirectional. Examples of this are provided throughout the disclosure.

Further, components of OR environment 100 that contain electrical and/or electromechanical elements can be coupled to a source of power via operating room infrastructure 118, Similarly, components of the OR environment 100 may be coupled to fluid and/or gas supplies via operating room infrastructure 118. For example, internal operating room display 102, external operating room display 104, imaging devices 106, robotic devices 108, patient devices 110, therapy consoles 112, and/or therapy devices 114 can be configured to receive electrical power, gas supply, water inflow and/or outflow supply, vacuum supply, or the like in any combination via operating room infrastructure 118.

Lastly, centralized operating theater controller 122 can be coupled to any equipment or components of the OR environment 100 via IT infrastructure 120. FIG. 1B illustrates an example centralized operating theater controller 122. In general, the centralized operating theater controller 122 can be configured to send and/or receive data to and/or from equipment (e.g., therapy consoles internal operating room display 102, external operating room display 104, imaging devices 106, robotic devices 108, patient devices 110, therapy consoles 112, therapy devices 114, equipment controls 116, and/or operating room infrastructure 118). Further, centralized operating theater controller 122 can be configured to processor data, infer information from data, and execute instructions to implement methods described herein.

It is noted that centralized operating theater controller 122 is depicted in FIG. 1A and FIG. 1B as a single components. However, in practice centralized operating theater controller 122 can be multiple components or a single component. Further, centralized operating theater controller 122 can be embodied (e.g., housed) in one of the other pieces of equipment depicts in FIG. 1A. For example, a one of the therapy consoles 112 can include hardware as depicted in FIG. 1B and can be configured as outlined herein to operate as centralized operating theater controller 122.

As depicted in FIG. 1B, centralized operating theater controller 122 can include computer system 124, input devices 126, output devices 128, and/or remote devices 130.

The computer system 124 may include processor 132 and a memory storage device 134 coupled to the processor 132 via a storage interface 136. In general, processor 132 can be processing circuitry configured to execute instructions stored on memory storage device 134. For example, processor 132 can be a central processing unit (CPU), a graphics processing unit, a machine learning (ML) processing unit, or a combination of these. The processor 132 may include specialized processing units such as integrated system (bus) controllers, memory management control units, floating point units, graphics processing units, neural processing units, digital signal processing units, etc. Processor 132 can be an off the shelf CPU or can be custom designs processing circuitry (e.g., an application specific integrated circuit (ASIC), or the like).

Memory storage device 134 may include computer-readable storage media or devices configured to store data. Such data can take a variety of forms or data structures. One form of such data is machine code (also referred to as “instructions”) that is executable by processor 132. In some examples, memory storage device 134 can be physical memory on which information or data readable by a processor (e.g., processor 132) may be stored. Thus, a computer-readable storage medium may store instructions for execution by one or more processors (e.g., processor 132, or the like) including instructions for causing the processor(s) to perform steps or stages consistent with the embodiments described herein.

The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media. Although depicted in FIG. 1B, memory storage device 134 need not be collocated with processor 132 and can for example, be accessible via a network.

In some embodiments, the storage interface 136 may be configured to connect to memory storage device 134 via memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Processor 132 can may be disposed in communication with input devices 126 and output devices 128 via I/O interface 138. The I/O interface 138 may employ communication protocols and/or methods such as, without limitation, audio, analog, digital, stereo, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), Ethernet, Bluetooth, cellular, etc. Using the I/O interface 138, computer system 124 may communicate with input devices 126 and output devices 128. In general, input devices 126 can be any control or input device used to provide input to equipment in the OR environment 100.

In some embodiments, input devices 126 can include devices configured to receive input from a user of the OR environment 100 (e.g., a physician, a nurse, an assistance, a technician, or the like). For example, input devices 126 can include wearable controls such as, watches, armbands, headphones, footwear, or the like. In another example, input devices 126 can include touch-based controls such as, foot pedals, switches, triggers, touch screens, buttons, or the like. In some examples, input devices 126 can include non-touch-based controls such as, voice activation controls, gesturebased controls, eye movement based controls, or the like. These various input devices implemented as input devices 126 can be equipment controls 116 described above or can be inputs to other computing equipment (e.g., imaging devices 106, robotic devices 108, patient devices 110, therapy consoles 112, therapy devices 114, etc.)

With some embodiments, output devices 128 can include internal operating room display 102 and/or external operating room display 104. With some embodiments, output devices 128 can include non-display outputs such as, audio output, flashing light output, haptic output, or the like. Further, in some examples, ones of input devices 126 and/or output devices 128 can be combined. For example, a touch screen display can be configured as both input devices 126 and output devices 128.

It is to be appreciated that although input devices 126 and output devices 128 are depicted as included (or packaged) with computer system centralized operating theater controller 122, they may not be explicitly part of centralized operating theater controller 122 but could be I/O devices of another computer system described herein with which centralized operating theater controller 122 is configured to communicate.

As noted, memory storage device 134 may store instructions executable by processor 132. This can include various types of instructions. For example, memory storage device 134 can store an operating system 144 and/or application instructions 146. Further, memory storage device 134 can store graphical instructions and elements 148 (e.g., user interface elements, graphical information elements, etc.) In various embodiments, the operating system 144 may facilitate resource management and operation of the computer system 124 and facilitate communicative coupling with input devices 126, output devices 128, and other equipment in the OR environment 100 coupled via IT infrastructure 120. Examples of operating systems include, without limitation, UNIX®, UNIX-like system distributions (E.G., BERKELEY SOFTWARE DISTRIBUTION® (BSD), FREEBSD®, NETBSD®, OPENBSD, etc.), LINUX® DISTRIBUTIONS (E.G., RED HAT®, UBUNTU®, KUBUNTU®, etc.), IBM® OS/2®, MICROSOFT WINDOWS®, APPLE® IOS®, GOOGLE™ ANDROID™, or the like.

The application instructions 146 may include instructions that when executed by the processor 132 cause centralized operating theater controller 122 to perform one or more techniques, steps, procedures, and/or methods described herein, such as to send and/or receive data to and/or from equipment of OR environment 100, process the data, infer information from ML models, execute algorithms based on the data, and send and/or receive control signals to and/or from the equipment in the OR environment 100.

The graphical instructions and elements 148 may include instructions that when executed by the processor 132 cause the processor 132 to facilitate rendering and display of information on displays in patient devices 110. The graphical instructions and elements 148 can also include the rendered graphical elements (or frames) to be displayed. For example, graphical instructions and elements 148 can be configured to provide cursors, icons, checkboxes, menus, scrollers, windows, widgets, etcetera. Such graphical instructions and elements 148 can provide an interface (e.g., analog, or digital) to equipment of the OR environment 100 or a display of settings, parameters, status, or other information related to the equipment.

In some embodiments, the processor 132 may be in communication with remote devices 130 via network interface 140 and communications network 142. Remote devices 130 can be any of a variety of computing devices (e.g., cloud computing resources, cloud storage resources, remote servers, remote sensors, remote workstations, etc.) The network interface 140 may enable communication between computer system 124 and remote devices 130 via the communications network 142. To that end, the network interface 140 may employ connection protocols including, without limitation, direct connect, Ethernet, Transmission Control Protocol/Internet Protocol (TCP/IP), token ring, Wi-Fi, etc. The communications network 142 can be implemented as one of the different types of networks, such as intranet or Local Area Network (LAN), Closed Area Network (CAN), the Internet, and such. The communications network 142 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 142 may include a variety of network devices, including routers, bridges, servers, computing devices, storage devices, etcetera.

Many pieces of equipment in OR environment 100 can include computing components or a computing system. For example, therapy consoles 112 often include computing systems including computing components like described above with respect to centralized operating theater controller 122 and particularly computer system 124. Accordingly, computing components of such devices are often discussed with reference to the components of computer system 124. However, this is done for convenience only. It is to be appreciated that these other computer systems many include some or all the components of computer system 124 and may include different components or additional components that are not shown in FIG. 1B. In general, however, such computer system will include a processor (e.g., like processor 132) and a memory storage device (e.g., like memory storage device 134) that includes instructions.

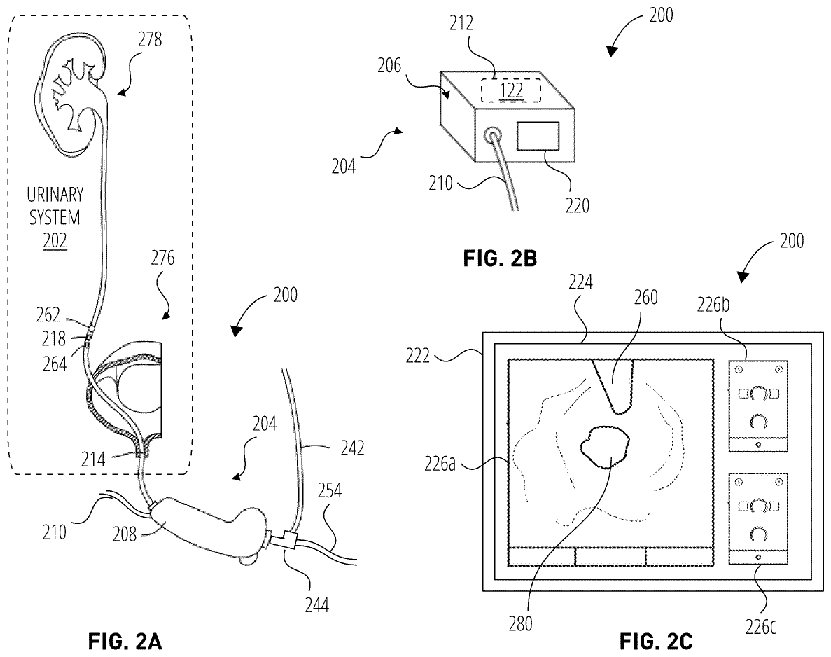

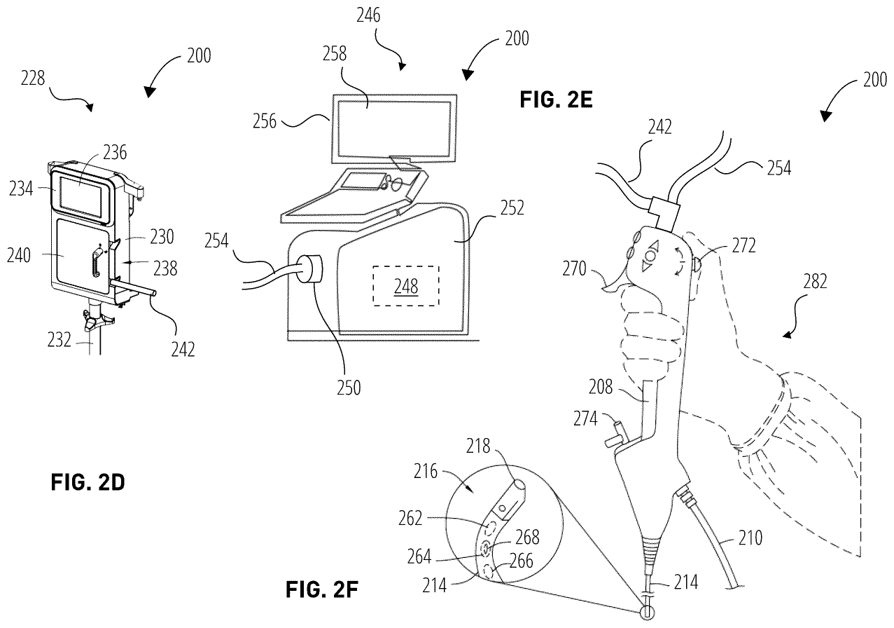

As noted above, OR environment 100 can be provisioned and implemented to perform urological procedures. For example, FIG. 2A to FIG. 2E illustrates an OR environment 200, which can be implemented using components from OR environment 100 described above to perform a urological procedure. OR environment 200 is described with respect to a lithotripsy procedure to treat urinary calculi (referred to as “stone”) in a urinary system 202. However, this is not intended to be limiting and OR environment 100 and/or OR environment 200 could be implemented to perform other urological procedures, such as, for example, percutaneous nephrolithotomy (PCNL), benign prostatic hyperplasia (BPH), transurethral resection of the prostate (TURP), etc.

OR environment 200 can be implemented with an endoscope 204, such as, a ureteroscope. Endoscope 204 can include an endoscope console 206 (e.g., one of therapy consoles 112), which can be configured to operate with an endoscope handle 208 (e.g., one of therapy devices 114). The endoscope console 206 and endoscope handle 208 can be coupled via connection cable 210. FIG. 2A, and FIG. 2F illustrate endoscope handle 208 in OR environment 200 while FIG. 2B illustrates endoscope console 206. Endoscope 204 can be coupled to a source of power via operating room infrastructure 118. Further, the connection cable 210 can be configured to provide power from endoscope console 206 to endoscope handle 208 and to provide exchange of data between endoscope console 206 and endoscope handle 208.

Endoscope console 206 can include computing system 212 (e.g., like computer system 124) which itself can include (or be coupled to) a display 220 (e.g., touch screen display, or the like). Endoscope console 206 can also include input and/or output devices (not shown), such as buttons, lights, switches, etc. Further, OR environment 200 can include computing components configured to operate as the centralized operating theater controller 122 described above with respect to FIG. 1A and FIG. 1B. With some embodiments, as shown, centralized operating theater controller 122 can be integrated into one of the therapy consoles 112. Accordingly, endoscope console 206 can include computing system 212 and computer system 124.

In some embodiments a single computing system (e.g., computer system 124, or the like) can be provided as part of endoscope console 206 and configured to operate as both computing system 212 and centralized operating theater controller 122. For example, FIG. 2B illustrates centralized operating theater controller 122 into integrated endoscope console 206 as part of computing system 212. However, this is not intended to be limiting and centralized operating theater controller 122 could be a separate computing system disposed in the housing of endoscope console 206 or could be integrated into another therapy consoles 112 provisioned in the OR environment 200 (e.g., theater display 222, fluidics unit 228, laser energy console 246, or the like). Or as depicted in OR environment 100, centralized operating theater controller 122 of OR environment 200 could be a stand-alone component provisioned in OR environment 200. In some embodiments, centralized operating theater controller 122 can be a cloud computing system (e.g., computing as a service (CaaS), or the like) accessible via communications network 142. In such as example, equipment in OR environment 200 (e.g., computing system 212 of 206, or the like) can include network interfaces (e.g., network interface 140) to enable communication with centralized operating theater controller 122 on communications network 142.

Operation of centralized operating theater controller 122 in OR environment 200 is described in greater detail below. However, in general, centralized operating theater controller 122 is configured to provide data communication between devices provisioned in OR environment 200. For example, centralized operating theater controller 122 can be configured to provide data communication between endoscope 204, display 220, fluidics unit 228, laser energy console 246, and their associated therapy devices 114, or any combination of these components. Further, centralized operating theater controller 122 can be configured to process such data as outlined herein (e.g., send and receive control signals between devices based on (or responsive to) the communicated data, execute algorithms on the communicated data as part of controlling operation of the components, or the like).

Endoscope 204 can include an elongated shaft 214 coupled to endoscope handle 208, which can be used to access a patient's bladder 276 and/or kidney 278. In such a procedure, the endoscope 204, and particularly, a distal end 216 of the elongated shaft 214 is inserted into the bladder 276 via the urethra and can be further inserted into the kidney 278 via the ureter, where it can be used to diagnose and/or treat a variety of problems in the urinary system 202. The endoscope 204 can include a camera 218 disposed on the distal end 216 of the elongated shaft 214. The camera 218 can be used to provide a visual feed on a display screen. For example, images captured by camera 218 can be rendered and displayed on display 220 of endoscope console 206. Additionally, OR environment 200 can be provided with several other displays (e.g., internal operating room display 102 and/or external operating room display 104) that can be configured to display images and/or video captured by camera 218 of endoscope 204. For example, centralized operating theater controller 122 can be configured, or rather, can include application instructions 146 stored in memory storage device 134 that when executed by processor 132 cause centralized operating theater controller 122 to receive data comprising indications of image frames captured by camera 218, process the image frames, and send the processed image frames to theater display 222 for display. Such communication can be facilitated by communicative connection between the equipment in OR environment 200 via IT infrastructure 120.

For example, FIG. 2C illustrates a theater display 222 (or operating theater display), in which is depicted a composite display 224 having a grouping of individual graphical elements 226a to 226c. For example, theater display 222 shown composite display 224 having graphical element 226a, 226b, and 226c where graphical element 226a depicts a view of an image captured by camera 218. It is to be appreciated that this view could be a live view or a recorded view and various examples of each are provided herein.

OR environment 200 can further include a fluidics unit 228 (e.g., as one of therapy consoles 112). Fluidics unit 228 can be coupled to endoscope 204 and called on to provide fluid flow to the distal end 216 of the elongated shaft 214. For example, fluidics unit 228 could be utilized to clear the visual field of the camera 218. Fluidics unit 228 can include a console 230. In some examples, console 230 can be mounted on pole 232 attached to a mobile base (not shown). In other examples, console 230 can be free standing, table mounted, or the like. Console 230 can include computing system 234 (e.g., like computer system 124) which itself can include a display 236 (e.g., touch screen display, or the like). Fluidics unit 228 can also include input and/or output devices (not shown), such as, buttons, lights, switches, etc.

Console 230 can include an interface (not shown) with connection sockets and/or busses to which computing system 234 can be communicatively coupled to centralized operating theater controller 122 via IT infrastructure 120. Such interface can also couple fluidics unit 228 to a source of power via operating room infrastructure 118. For example, a connection cable (not shown) could couple computing system 234 to centralized operating theater controller 122 in endoscope console 206 (e.g., via IT infrastructure 120, or the like) and couple fluidics unit 228 to power provided by operating room infrastructure 118.

Fluidics unit 228 can include a pump 238 (disposed in console 230). The pump can be configured to provide fluid flow when requested by the user (e.g., via the endoscope handle 208, or the like). Fluidics unit 228 can be configured to operate with a cassette and tubing set 242 (e.g., one of therapy devices 114, or the like). The cassette and tubing set 242 can be disposed in console 230 via door 240. Further, the cassette and tubing set 242 can be coupled to a source of fluid (not shown) and to the endoscope handle 208 (e.g., via fluid port 244 as shown in FIG. 2A, or the like). In some examples, the fluid source can be saline bags, or the like.

The computing system 234 can control pump 238 (e.g., responsive to input from endoscope 204, endoscope handle 208, responsive to sensor(s) output, responsive to control signals from centralized operating theater controller 122, or the like) to cause fluid to flow to the distal end 216 of the elongated shaft 214 via a working channel or dedicated fluid channel (not shown) in the elongated shaft 214. Additionally, in some embodiments, fluidics unit 228 can include a heater and/or a chiller to heat and/or cool the fluid supplied to the treatment site via the elongated shaft 214. Fluid flow to the treatment site (e.g., body cavity, or the like) in the urinary system 202 where the stone 280 is located affects the pressure inside the body cavity. This pressure is referred to herein as intraluminal pressure (ILP).

During an example lithotripsy procedure, blood and/or debris may be present in the body cavity, which may negatively affect image quality captured by the endoscope 204. Fluid flow (e.g., irrigation fluid flow) from fluidics unit 228 may be used to flush the body cavity to improve image the quality. Further, as laser energy (described below) can be used to fragment, ablate, dust, or otherwise treat the stone, heat may be generated at the treatment site. Fluid flow can be used to control the temperature of the treatment site to avoid damage or injury to adjacent tissue.

OR environment 200 can further include a laser energy console 246 (e.g., as one of therapy consoles 112) provisioned in OR environment 200. Continuing with the example discussed above where OR environment 200 is provisioned for a lithotripsy procedure, laser energy console 246 could be a medical laser console, such as, a Holmium (Ho) laser or a Thulium (Tm) fiber laser console. As another example, laser energy console 246 could be a tissue ablation console (e.g., electronic ablation, RF ablation, etc.). With yet another example, laser energy console 246 could be a laser morcellator. With some embodiments, OR environment 200 could be provisioned with multiple laser energy consoles 246 (e.g., a morcellator and stone dusting console, or the like). Further, although not shown, OR environment 200 could include other consoles appropriate for the procedure to be performed in OR environment 200.

Laser energy console 246 can include a laser generator 248 and an optical coupler 250, both disposed in a housing 252. The laser generator 248 can be configured to generate laser energy appropriate for treating a target tissue (e.g., stone 280). A treatment fiber 254 (e.g., one of therapy devices 114, or the like) can be coupled to the laser generator 248 via the optical coupler 250. In some embodiments, laser generator 248 can comprise multiple light sources (e.g., a treatment beam, multiple treatment beams, an aiming beam, a diagnostic beam, etc.). Further, laser generator 248 can often include various optical components and sensors configured to measure characteristics or qualities of the laser energy and its effect on the stone 280, or adjacent tissue.

Laser energy console 246 can include computing system 256 (e.g., like computer system 124) which itself can include a display 258 (e.g., touch screen display, or the like). Laser energy console 246 can also include input and/or output devices (not shown), such as, buttons, lights, switches, foot pedals, etc.

Housing 252 can include an interface (not shown) with connection sockets and/or busses to which computing system 256 can be communicatively coupled to centralized operating theater controller 122 via IT infrastructure 120. Such interface can also couple laser energy console 246 to a source of power via operating room infrastructure 118. For example, a connection cable (not shown) could couple computing system 256 to centralized operating theater controller 122 in endoscope console 206 (e.g., via IT infrastructure 120, or the like) and couple laser energy console 246 to power provided by operating room infrastructure 118.

During an example lithotripsy procedure, the treatment fiber 254 can be inserted into port 244 of endoscope handles 208 and pushed through a working channel (not shown) of elongated shaft 214 such that a distal end 260 of the treatment fiber 254 can be positioned proximate to stone 280 in urinary system 202. For example, graphical element 226a depicts an image captured by camera 218 of endoscope 204 in which the distal end 260 of the treatment fiber 254 and stone 280 are shown in the urinary system 202. Laser generator 248 can generate laser energy, which is optically coupled to treatment fiber 254 via the optical coupler 250. The laser energy is conveyed through the treatment fiber 254 and emitted from the distal end 260, where it may be incident on stone 280 to cause the stone 280 to be treated (e.g., ablated, fragmented, dusted, etc.)

The computing system 256 can control laser generator 248 (e.g., responsive to input from endoscope 204, endoscope handle 208, responsive to an input device like a foot pedal, responsive to sensor(s) output, responsive to control signals from centralized operating theater controller 122, or the like) to cause the laser generator 248 to generate laser energy having parameters appropriate for the treatment to be generated. Examples of this are described in greater detail below.

In some embodiments, the working channel in which the treatment fiber 254 is inserted is different from the working channel through which fluid supplied by fluidics unit 228 flows. With some embodiments, the working channel in which the treatment fiber 254 is inserted in the same working channel through which fluid supplied by fluidics unit 228 flows.

A user (e.g., physician, a nurse, an assistance, or the like) of OR environment 200 can configure (e.g., enter treatment therapy details, or the like) via the computing components of each respective one of therapy consoles 112 provisioned in OR environment 200. For example, a user can configure endoscope 204 via computing system 212, configure fluidics unit 228 via computing system 234, and configure laser energy console 246 via computing system 256. As another example, a user can configure individual ones of the components of OR environment 200 via centralized operating theater controller 122.

Further, a user can perform a treatment via one of more of the therapy devices 114 described above. For example, endoscope 204 includes endoscope handle 208, which is depicted in use by a user 282 in FIG. 2F. As outlined above, the endoscope handle 208 can be fluidly coupled to fluidics unit 228 via cassette and tubing set 242. Further, a treatment fiber 254 can be disposed through endoscope handle 208 and into urinary system 202. It is to be appreciated that although FIG. 2F depicts a user 282 manipulating endoscope handle 208 during a procedure in OR environment 200, other embodiments may provide robotic, non-manual, or non-touch-based control of devices, such as, endoscope handle 208. Further, it is to be appreciated that the user need not be in the operating theater to control the therapy consoles 112 (e.g., see FIG. 3A).

In some embodiments, the endoscope 204 may include one or more sensors, which can be disposed proximate the distal end 216 of the elongate elongated shaft 214. For example, FIG. 2F depicts pressure sensor 262 at the distal end 216 of the elongate elongated shaft 214. Pressure sensor 262 can be configured to measure an intraluminal pressure (ILP) within the treatment site (see FIG. 2A). The endoscope 204 may also include other sensors such as, for example, a temperature sensor 264, a grating 266 (e.g., a Fiber Bragg grating, or the like) to detect stresses, and/or an antenna or electromagnetic sensor 268 (e.g., a position sensor).

Further, as noted, the endoscope 204 includes at least one camera 218 disposed at the distal end 216 of the elongated shaft 214 to provide a visual feed (e.g., as shown in graphical element 226a, or the like) to the user. The endoscope handle 208 can have a fluid flow on/off switch 270, which allows the user 282 to control when fluid is flowing through the elongated shaft 214 and into the treatment site. The endoscope handle 208 may further include other buttons 272 that perform other functions (e.g., control other devices provisioned in OR environment 200, or the like). For example, in some embodiments, the endoscope handle 208 may include buttons 272 to control the temperature of the fluid. In some embodiments, the endoscope handle 208 may also include a drainage port 274, which may be connected to a drainage system (e.g., of operating room infrastructure 118) and can be configured to provide a path for return flow of fluid from the treatment site.

As indicated above, all components of the urological OR suite need not reside in the operating theater. For example, equipment and user of OR environment 100 or OR environment 200 could be in different room, buildings, sites, or geographic locations. FIG. 3A depicts an OR environment 300 having multiple rooms. In some embodiments, OR environment 300 can be implemented to provide remote proctorship and/or telesurgery. OR environment 300 provides an advantage in that highly trained specialists can be utilized to observe, supervise, train, troubleshoot, and/or otherwise facilitate procedures across OR suites without having to travel to each suite.

FIG. 3A depicts OR environment 300 with operating theater 304, observation room 306, and remote room 308. In general, operating theater 304 is the room where the patient and the bulk of the equipment used to monitor and treat the patient are located. Observation room 306 can be a room proximate too but separate from operating theater 304. For example, observation room 306 can be outside the sterile field, separated from operating theater 304 by a glass wall or window, or the like. Remote room 308 can be a room in the same building as operating theater 304 and observation room 306, in another building from operating theater 304 and observation room 306, or even in another physical or geographic location (e.g., different facility site, different city, different country, partner facility site, etc.)

Communication and interoperability between the equipment in rooms of OR environment 300 is facilitated by centralized operating theater controller 302. Centralized operating theater controller 302 can be implemented as centralized operating theater controller 302 and can include all the components, structure, and features with which centralized operating theater controller 302 is described and attributed herein. As depicted, centralized operating theater controller 302 is provided as a cloud accessible computing system (e.g., in communications network 142). However, centralized operating theater controller 302 could be provided as part of endoscope 204 like described above in FIG. 2B, or any other piece of equipment in OR environment 300.

Operating theater 304, observation room 306, and remote room 308 can be connected via IT infrastructure 120, which can include communications network 142. As such, centralized operating theater controller 302 can communicate with equipment in each room.

OR environment 300 is described with reference to the OR environment 200 described above for consistency and clarity. However, OR environment 300 could be provisioned with equipment other than described herein. Continuing with the example lithotripsy procedure described above, operating theater 304 can include patient bed 310, patient monitor 312, endoscope 204, fluidics unit 228, laser energy console 246, and audio-visual communication equipment 316a (A/V equipment). Operating theater 304 can also be provisioned with theater display 222, equipment controls 314a, and/or robotic devices 108. However, OR environment 300 could be implemented where 304 is not provisioned with theater display 222 and equipment controls 314a, for example, where users needing theater display 222 and equipment controls 314a are not located in operating theater 304. As another example, operating theater 304 could be provisioned with robotic devices 108 where control of equipment in operating theater 304 (e.g., endoscope 204, fluidics unit 228, laser energy console 246, or the like) from remote room 308 is implemented.

Remote room 308 can include remote computing system 318 (e.g., like computer system 124, or the like) including remote display 320 and A/V equipment 316b. Remote room 308 can further include equipment controls 314b. For example, where remote room 308 is used to control (e.g., telesurgery, or the like) equipment in operating theater 304, remote room 308 can include equipment controls 314b. Observation room 306 can include A/V equipment 316c and may also include equipment controls 314c and/or observation display 322.

Equipment controls 314a, 314b, and 314c can include any of equipment controls 116 described herein. A/V equipment 316a, 316b, and 316c can be cameras, microphones, or other equipment configured to provide a view of the procedure and/or communication between rooms. For example, A/V equipment 316a, 316b, and 316c can include a microphone and speaker (e.g., fixed in place in the respective room, wearable, etc.) to enable audio communication between rooms. A/V equipment 316a can include a camera positioned to provide a view of the patient and endoscope handle 208, treatment fiber 254, cassette and tubing set 242, and/or other therapy devices 114. With some embodiments, A/V equipment 316b and 316c can include a camera arranged to provide views of the occupants of each respective room.

Further, various therapy devices 114 can be provided in operating theater 304, for example, endoscope handle 208, cassette and tubing set 242, and treatment fiber 254 can be provided in operating theater 304.

During operation, centralized operating theater controller 302 can provide for monitoring, parameter adjustment, and/or control of equipment in operating theater 304 by users in operating theater 304, observation room 306, and/or remote room 308. For example, centralized operating theater controller 302 can provide monitoring of patient bed 310, patient monitor 312, endoscope 204, fluidics unit 228, and laser energy console 246 via observation display 322 in observation room 306. Further, centralized operating theater controller 302 can provide control of endoscope 204, fluidics unit 228, and laser energy console 246 via equipment controls 314b in remote room 308. For example, a specialist physician can be positioned in endoscope handle 208 can assigned the responsibility of controlling endoscope 204, fluidics unit 228, and laser generator 248 while a nurse can be positioned in operating theater 304 and assigned the responsibility of monitoring patient bed 310 and patient monitor 312. As such, centralized operating theater controller 302 can be configured to control (e.g., robotic devices 108, or the like) equipment in operating theater 304 from inputs and/or control signals received from equipment controls 314b in remote room 308.

FIG. 3B illustrates the centralized operating theater controller 302 shown in FIG. 3A in greater detail. It is to be appreciated that this figure may not depict all elements of centralized operating theater controller 302. For example, elements such as the operating system 144, interconnects, or the like are omitted for clarity. Centralized operating theater controller 302 includes at least a processor 132 and memory storage device 134. In general, memory storage device 134 stores instructions (e.g., application instructions 146, or the like) executable by the processor 132, which when executed cause the centralized operating theater controller 302 to provide remote proctoring and/or telesurgery functionality of any OR environment described herein.

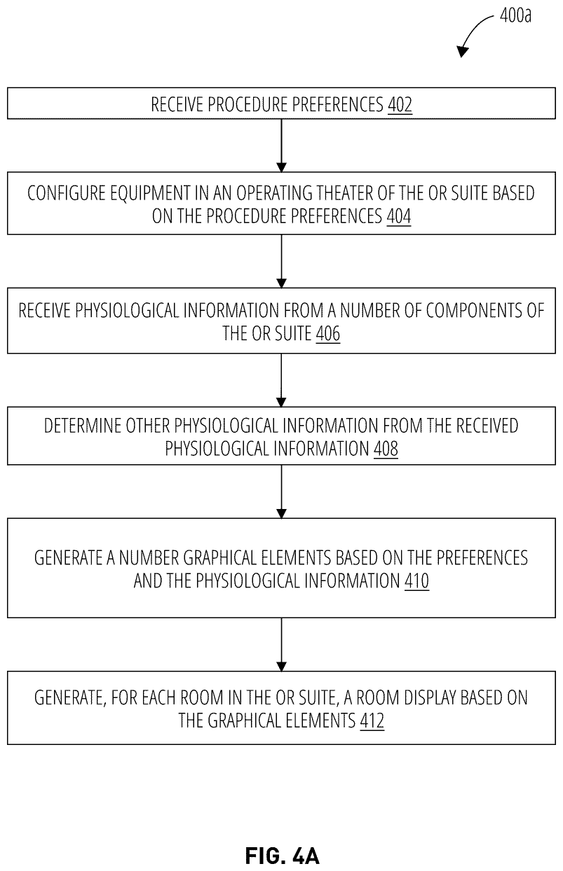

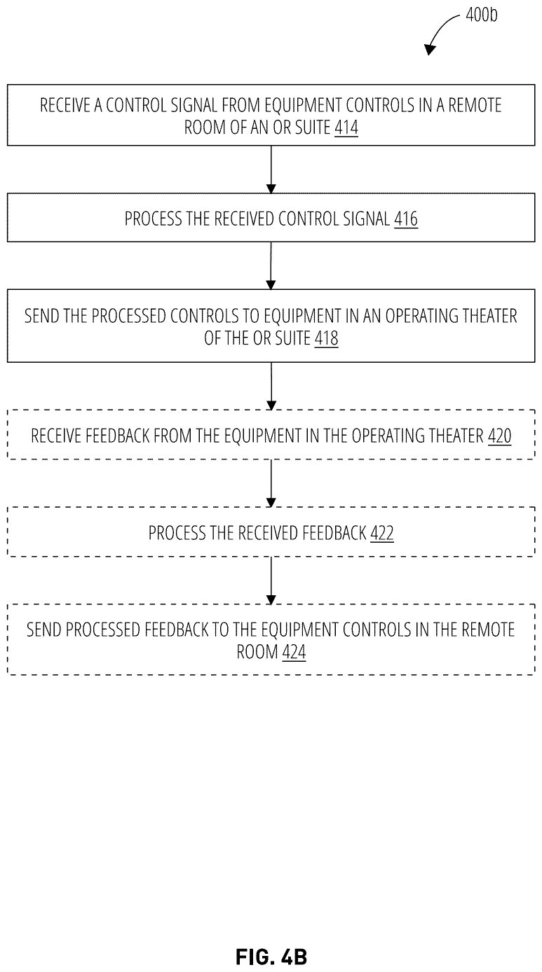

Centralized operating theater controller 302 is described with reference to FIG. 4A and FIG. 4B. FIG. 4A and FIG. 4B illustrate logic flows 400a and 400b, respectively. These logic flows can be carried out by centralized operating theater controller 302 to provide remote proctoring and/or telesurgery. In some embodiments, centralized operating theater controller 302 can carry out logic flows 400a and 400b simultaneously.

Logic flow 400a can begin at block 402. At block 402 “receive procedure preferences” preferences for the treatment procedure being carried out can be received. In some examples, these preferences can be based on the type of procedure and equipment provisioned in the OR suite. In further examples, these preferences can be based on a physician in the OR suite, established clinic preferences, or the like. Processor 132 can execute application instructions 146 to cause centralized operating theater controller 302 to receive procedure preferences 324 (e.g., from a data center, from local storage, from a cloud-based storage location, from equipment controls 314a, equipment controls 314b, or the like). For example, processor 132 can execute application instructions 146 to receive procedure preferences 324 from user (e.g., proctor, physician, etc.) in remote room 308 via equipment controls 314b.

Continuing to block 404 “configure equipment in an operating theater of the OR suite based on the procedure preferences” equipment in the operating theater of OR environment 300 can be configured based on the procedure preferences. Processor 132 can execute application instructions 146 to cause centralized operating theater controller 302 to configure or otherwise adjust settings on equipment (e.g., endoscope 204, fluidics unit 228, laser energy console 246, etc.) based on the procedure preferences 324. For example, procedure preferences 324 may be parameters for laser energy to be generated by laser energy console 246. As such, processor 132 can execute application instructions 146 to generate configuration control signals 326 and configuration control signals 326 to laser energy console 246 to cause laser energy console 246 to be placed in a configuration wherein laser energy having the desired parameters will be generated. As another example, procedure preferences 324 may be parameters for fluid flow supplied by fluidics unit 228 and/or ILP. As such, processor 132 can execute application instructions 146 to generate configuration control signals 326 and send configuration control signals 326 to fluidics unit 228 to cause fluidics unit 228 to be placed in a configuration wherein fluid from cassette and tubing set 242 will flow according to the parameters.

Continuing to block 406 “receive physiological information from a number of components of the OR suite” physiological data can be received from equipment of the OR environment 300. Processor 132 can execute application instructions 146 to cause centralized operating theater controller 302 to receive physiological information 328a from devices and/or consoles of OR environment 300. For example, processor 132 can execute application instructions 146 to receive data (e.g., an information element, sensor output signals, or the like) comprising indications of an intensity of laser energy (e.g., diagnostic energy, aiming energy, treatment energy, or the like) generated by laser generators 248. With some embodiments, processor 132 can execute application instructions 146 to receive physiological information 328a from computing system 256 of laser energy console 246. With some embodiments, processor 132 can execute application instructions 146 to determine the intensity based on signals received from sensors (not shown) of laser energy console 246 where the sensors are configured to measure qualities and/or characteristics of the laser energy.

In some embodiments, processor 132 can execute application instructions 146 to receive physiological information 328a comprising indications of turbidity and/or clarity of scene(s) captured by camera 218 of endoscope 204. In some embodiments, processor 132 can execute application instructions 146 to receive captured scene information 330 comprising image frames of the scene(s). In some embodiments, processor 132 can execute application instructions 146 to receive physiological information 328a comprising indications of an ILP, flow rate of fluidics unit 228, or both.

In some embodiments, processor 132 can execute application instructions 146 to receive physiological information 328a comprising indications of a distance between the distal end 260 of the treatment fiber 254 and the stone 280, a composition of the stone 280, and/or a texture of the stone 280. With some embodiments, laser energy console 246 can be configured to measure this distance and computing system 256 can communicate the distance to centralized operating theater controller 302. With some embodiments, laser energy console 246 can be configured to determine the composition and/or texture of the stone 280 and computing system 256 can communicate the composition and/or texture to centralized operating theater controller 302.

In some embodiments, laser energy console 246 can be configured to measure characteristics of the laser energy and treatment environment (e.g., intensity of laser energy, intensity of reflected laser energy, intensity of autofluorescence emitted responsive to incidence of laser energy on the stone, etc.) Processor 132 can execute application instructions 146 to receive this information as physiological information 328a and derive the distance between the distal end 260 and the stone 280, the composition of the stone 280, and/or the texture of the stone 280 based on physiological information 328a.

In some embodiments, processor 132 can execute application instructions 146 to receive physiological information 328a comprising indications of a size of the stone 280 from endoscope 204. For example, processor 132 can execute application instructions 146 to receive captured scene information 330 where the stone 280 is represented. As another example, processor 132 can execute application instructions 146 to receive radiological image physiological information 328a from radiological imaging devices 106 (e.g., an ultrasound, an x-ray, or the like) where the stone 280 is represented in the radiological image physiological information 328a.

Continuing to block 408 “determine other physiological information from the received physiological information” other physiological data can be determined (e.g., derived, inferred, or the like) from physiological information 328a received at block 406. Processor 132 can execute application instructions 146 to cause centralized operating theater controller 302 to derive and/or infer other physiological information 328b from physiological information 328a.

In some embodiments, memory storage device 134 can store a models 334. Models 334 can comprise algorithms, functions, and/or trained machine learning (ML) models configured to derive and/or infer physiological information 328b from physiological information 328a.

For example, processor 132 can execute application instructions 146 to determine, using models 334, a turbidity and/or clarity of captured scene information 330 from physiological information 328a and/or captured scene information 330. As another example, processor 132 can execute application instructions 146 to determine, using models 334, a distance between the distal end 260 and the stone 280, a composition of the stone 280, a texture of the stone 280, and/or a size of the stone (e.g., viewed from the camera 218 and/or viewed radiologically) from physiological information 328a, captured scene information 330, and/or radiological image information 332.

Continuing to block 410 “generate a number of graphical elements from the physiological information” several graphical elements 336 can be generated from the physiological information 328a and 328b (including the captured scene information 330 and radiological image information 332). In general, processor 132 can execute application instructions 146 to generate graphical elements 336 representative of physiological information 328a and 328b associated with each component in the OR suite (e.g., endoscope 204, fluidics unit 228, laser energy console 246, etc.) Further, the graphical elements 336 visually depict represented information using images, text, icons, colors, movement, or the like. With some embodiments, the graphical elements 336 can be like “alerts” or pop-up graphics.

For example, processor 132 can execute application instructions 146 to generate graphical elements 336 comprising an indication of the captured scene information 330. As another example, processor 132 can execute application instructions 146 to generate graphical elements 336 comprising an indication of an ILP and/or flow rate. As another example, processor 132 can execute application instructions 146 to generate graphical elements 336 comprising an indication of a size of the stone 280 or a composition of the stone 280. As another example, processor 132 can execute application instructions 146 to generate graphical elements 336 comprising an indication of an intensity of the laser energy incidence of the stone 280, a distance between the distal end 260 and the stone 280, or the like.

In some embodiments, the information visually represented in graphical elements 336 is based on procedure preferences 324. For example, the information is based on the procedure type and equipment provisions, which can be indicated in procedure preferences 324. As a further example, a first physician (e.g., in operating theater 304) may prefer to pay attention to a first subset of the physiological information 328a and 328b while another physician (e.g., in remote room 308) may prefer to pay attention to a second subset of the physiological information 328a and 328b. In general, the first and second subsets may overlap, but this is not required.

Continuing to block 412 “generate, for each room of the OR suite, a room display based on the graphical elements” room displays 338 comprising multiple graphical elements 336 can be generated for each room of OR environment 300. For example, a display of room displays 338 can be generated for theater display 222, remote display 320, and/or observation display 322. As outlined above, each of room displays 338 may comprise different combinations of graphical elements 336 depending upon which physical display (e.g., theater display 222, remote display 320, observation display 322, or the like) the display is to be displayed on. In some embodiments, the processor 132 can execute application instructions 146 to generate room displays 338 from graphical elements 336 based on procedure preferences 324. For example, a user of operating theater 304 viewing theater display 222 can specific which graphical elements 336 are preferred or desired to be visible on theater display 222. These presences can be dictated in procedure preferences 324 and the processor 132 can execute application instructions 146 to generate a display of room displays 338 for theater display 222 from graphical elements 336 based on the procedure preferences 324 for theater display 222. As another example, a user of remote room 308 viewing remote display 320 can specific which graphical elements 336 are preferred or desired to be visible on remote display 320. These presences can be dictated in procedure preferences 324 and the processor 132 can execute application instructions 146 to generate a display of room displays 338 for remote display 320 from graphical elements 336 based on the procedure preferences 324 for remote display 320.

Logic flow 400b can begin at block 414. At block 414 “receive a control signal from equipment controls in a remote room of an OR suite” control signals from equipment controls in a remote room of an OR suite can be received. Processor 132 can execute application instructions 146 to cause centralized operating theater controller 302 to receive actuation control signals 340 from equipment controls 314b in remote room 308. For example, during operation, a user in remote room 308 can actuate equipment controls 314b and equipment controls 314b can generate actuation control signals 340, which can be communicated to and received by centralized operating theater controller 302 (e.g., via IT infrastructure 120, or the like).

Continuing to block 416 “process the received control signal” the received control signals can be processed into processed control signals. For example, processor 132 can execute application instructions 146 to process actuation control signals 340 to generate processed control signals 342. In general, processed control signals 342 are versions of actuation control signals 340 configured, processed, or otherwise translated for communication to equipment in operating theater 304 (e.g., robotic devices 108, endoscope 204, endoscope handle 208, fluidics unit 228, laser energy console 246, treatment fiber 254, or the like).

Continuing to block 418 “send the processed controls to equipment in an operating theater of the OR suite” the processed control signals can be sent to equipment in the operating theater of the OR suite associated with the remote room for which the control signals were received. For example, processor 132 can execute application instructions 146 to send processed control signals 342 to equipment in operating theater 304. For example, actuation control signals 340 may be directed to laser energy console 246 and specify actuation or initiation of laser energy generation. As such, processor 132 can execute application instructions 146 to send processed control signals 342 to laser energy console 246 to cause laser energy console 246 to generate laser energy. As another example, procedure preferences 324 may specify a desire for fluid flow at the treatment site. As such, processor 132 can execute application instructions 146 to send processed control signals 342 to fluidics unit 228 to cause fluidics unit 228 to pump (e.g., via pump 238) fluid through cassette and tubing set 242. With some embodiments, blocks block 416 and block 418 can be initiated responsive to receiving a control signal or signals at block 414.

Logic flow 400b can optionally include blocks 420 to block 424. Where logic flow 400b includes these blocks, logic flow 400b can continue from block 418 to block 420. At block 420 “receive feedback from the equipment in the operating theater” feedback signals from equipment in the operating theater of the OR suite can be received. Processor 132 can execute application instructions 146 to cause centralized operating theater controller 302 to receive feedback signals 344 from equipment (e.g., endoscope 204, endoscope handle 208, fluidics unit 228, laser energy console 246, or the like) in remote room 308.

Continuing to block 422 “process the received feedback” the feedback signals can be processed into processed feedback signals. For example, processor 132 can execute application instructions 146 to process feedback signals 344 to generate processed feedback signals 346. In general, processed feedback signals 346 are versions of feedback signals 344 configured, processed, or otherwise translated for communication to equipment controls 314b in remote room 308.

Continuing to block 424 “send processed feedback to the equipment controls in the remote room” the processed feedback signals can be sent to the equipment controls in the remote room of the OR suite. For example, processor 132 can execute application instructions 146 to send processed feedback signals 346 to equipment controls 314b in remote room 308. With some embodiments, blocks block 422 and block 424 can be initiated responsive to receiving a feedback at block 420.

As described herein, an OR suite can include multiple displays. For example, the OR environment 200 described above provisioned for a lithotripsy procedure has at least 4 displays, the main theater display 222, the display 220 for the endoscope 204, the display 236 for the fluidics unit 228, and the display 258 for the laser energy console 246. Further, it is to be appreciated that this does not include patient specific devices (e.g., vital monitoring devices, or the like) and anesthesia devices. To that end, the present disclosure provides an OR suite configured to centralize the display of information relevant to the procedure or therapy with which the OR suite is provisioned.

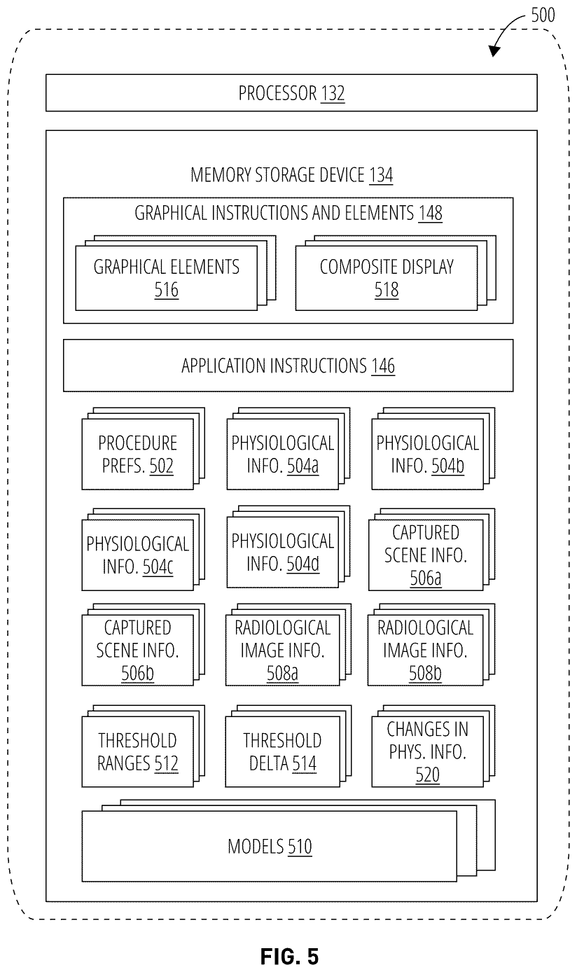

FIG. 5 illustrates a centralized operating theater controller 500, which can be implemented as centralized operating theater controller 122 and/or centralized operating theater controller 302 discussed above. It is to be appreciated that this figure does not depict all elements of centralized operating theater controller 500. For example, elements such as the operating system 144, interconnects, or the like are omitted for clarity. Centralized operating theater controller 500 and particularly an OR suite in which centralized operating theater controller 500 is implemented provides an advantage over conventional OR suites. For example, in each physical location of the OR suite, a user (e.g., a physician) can have a single monitor or display configured to provide relevant information from all or any desired combination of equipment (e.g., imaging devices 106, robotic devices 108, patient devices 110, therapy consoles 112, and/or therapy devices 114) provisioned in the OR suite and application for the therapy or procedure being conducted.

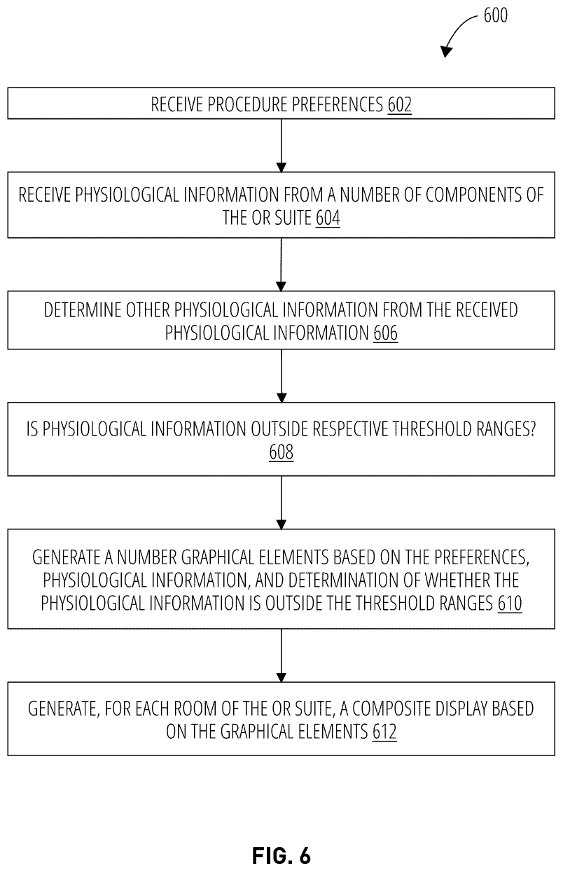

Centralized operating theater controller 500 includes at least a processor 132 and memory storage device 134. In general, memory storage device 134 stores instructions (e.g., application instructions 146, or the like) executable by the processor 132, which when executed cause the centralized operating theater controller 500 to provide a centralized display as described herein. Centralized operating theater controller 500 is described with reference to OR environment 200 and an example lithotripsy procedure for clarity of presentation. However, it is to be appreciated that centralized operating theater controller 500 could be implemented to provide centralized display of information for any of a variety of urological procedures. Further, detailed elements of centralized operating theater controller 500 are described with reference to FIG. 6. FIG. 6 illustrates logic flow 600, which can be carried out by centralized operating theater controller 500 to provide a centralized display of information.

Logic flow 600 can begin at block 602. At block 602 “receive procedure preferences” preferences for the treatment procedure being carried out can be received. In some examples, these preferences can be based on the type of procedure and equipment provisioned in the OR suite. In further examples, these preferences can be based on a physician in the OR suite, established clinic preferences, or the like. Processor 132 can execute application instructions 146 to cause centralized operating theater controller 500 to receive procedure preferences 502 (e.g., from a data center, from local storage, from a cloud-based storage location, or the like). For example, processor 132 can execute application instructions 146 to access remote devices 130 and retrieve procedure preferences 502 associated with the OR suite, procedure, and physician. Procedure preferences 502 are described in greater detail below.

Continuing to block 604 “receive physiological information from a number of components of the OR suite” physiological data can be received from components of the OR environment 200. Processor 132 can execute application instructions 146 to cause centralized operating theater controller 500 to receive physiological information 504a from devices and/or consoles of OR environment 200. For example, processor 132 can execute application instructions 146 to receive data (e.g., an information element, sensor output signals, or the like) comprising indications of an intensity of laser energy (e.g., diagnostic energy, aiming energy, treatment energy, or the like) generated by laser generators 248. With some embodiments, processor 132 can execute application instructions 146 to receive physiological information 504a from computing system 256 of laser energy console 246. With some embodiments, processor 132 can execute application instructions 146 to determine the intensity based on signals received from sensors (not shown) of laser energy console 246 where the sensors are configured to measure qualities and/or characteristics of the laser energy.

In some embodiments, processor 132 can execute application instructions 146 to receive physiological information 504a comprising indications of turbidity and/or clarity of scene(s) captured by camera 218 of endoscope 204. In some embodiments, processor 132 can execute application instructions 146 to receive captured scene information 506a comprising image frames of the scene(s). In some embodiments, processor 132 can execute application instructions 146 to receive physiological information 504a comprising indications of an ILP, flow rate of fluidics unit 228, or both.

In some embodiments, processor 132 can execute application instructions 146 to receive physiological information 504a comprising indications of a distance between the distal end 260 of the treatment fiber 254 and the stone 280, a composition of the stone 280, and/or a texture of the stone 280. With some embodiments, laser energy console 246 can be configured to measure this distance and computing system 256 can communicate the distance to centralized operating theater controller 500. With some embodiments, laser energy console 246 can be configured to determine the composition and/or texture of the stone 280 and computing system 256 can communicate the composition and/or texture to centralized operating theater controller 500.

In some embodiments, laser energy console 246 can be configured to measure characteristics of the laser energy and treatment environment (e.g., intensity of laser energy, intensity of reflected laser energy, intensity of autofluorescence emitted responsive to incidence of laser energy on the stone, etc.) Processor 132 can execute application instructions 146 to receive this information as physiological information 504a and derive the distance between the distal end 260 and the stone 280, the composition of the stone 280, and/or the texture of the stone 280 based on physiological information 504a.

In some embodiments, processor 132 can execute application instructions 146 to receive physiological information 504a comprising indications of a size of the stone 280 from endoscope 204. For example, processor 132 can execute application instructions 146 to receive captured scene information 506a where the stone 280 is represented. As another example, processor 132 can execute application instructions 146 to receive radiological image information 508a from radiological imaging devices 106 (e.g., an ultrasound, an x-ray, or the like) where the stone 280 is represented in the radiological image information 508a.

Continuing to block 606 “determine other physiological information from the received physiological information” other physiological data can be determined (e.g., derived, inferred, or the like) from physiological data received at block 604. Processor 132 can execute application instructions 146 to cause centralized operating theater controller 500 to derive and/or infer other physiological information 504b from physiological information 504a.

In some embodiments, memory storage device 134 can store a models 510. Models 510 can comprise algorithms, functions, and/or trained machine learning (ML) models configured to derive and/or infer physiological information 504b from physiological information 504a.

For example, processor 132 can execute application instructions 146 to determine, using models 510, a turbidity and/or clarity of captured scene information 506a from physiological information 504a and/or captured scene information 506a. As another example, processor 132 can execute application instructions 146 to determine, using models 510, a distance between the distal end 260 and the stone 280, a composition of the stone 280, a texture of the stone 280, and/or a size of the stone (e.g., viewed from the camera 218 and/or viewed radiologically) from physiological information 504a, captured scene information 506a, and/or radiological image information 508a.

Continuing to block 608 “is physiological information outside respective threshold ranges?” a determination of whether the physiological information is outside respective threshold ranges is made. Processor 132 can execute application instructions 146 to determine whether physiological information 504a and 504b are outside (or within) threshold ranges 512 and store the determinations as threshold delta 514 in memory storage device 134. With some embodiments, threshold ranges 512 can be set at the factor by the medical device manufacturer of the component. In other embodiments, threshold ranges 512 can be configured by a user (e.g., as part of procedure preferences 502, or the like). In yet other embodiments, threshold ranges 512 can be dynamically set based on physiological information 504a and 504b. That is, a range of threshold ranges 512 for one value of physiological information 504a or 504b can be dynamically set (e.g., during a procedure) based on other values of physiological information 504a and/or 504b. Accordingly, threshold ranges 512 can include any combination of pre-set thresholds, configurable thresholds, and dynamically set thresholds. As used herein, the term range is to mean either a range bound of both ends (e.g., between 0 and 1) and a range bound on only one end (e.g., less than or equal to 0 or greater than or equal to zero). Accordingly, as used herein the term “outside” the threshold range is intended to mean outside the range (e.g., less than the lower bound or greater than the upper bound, where bound on both ends, less than the bound when bound on a lower end, or greater than the bound when bound on a higher end).

In other examples, the term threshold range is used to mean a specific value or characteristic of physiological information 504a and/or 504b (e.g., composition, texture type, or the like). Further, in some embodiments, multiple thresholds can be specified for some values and/or characteristics of physiological information 504a and/or 504b.

For example, processor 132 can execute application instructions 146 to determine whether a composition of the stone 280 (as indicated in physiological information 504a and/or 504b) is outside (e.g., different from) a composition specified by threshold ranges 512. As another example, processor 132 can execute application instructions 146 to determine whether the size of the stone 280 (as indicated in physiological information 504a and/or 504b) is less than a threshold size (e.g., passable size, size small enough to retrieve, or the like). As another example, processor 132 can execute application instructions 146 to determine whether the distance between the distal end 260 of the treatment fiber 254 and the stone 280 (as indicated in physiological information 504a and/or 504b) is greater than a threshold distance. With some examples, there are multiple thresholds provided for the distance between the distal end 260 and the stone 280 in threshold ranges 512. In such an example, processor 132 can execute application instructions 146 to determine which, if any, threshold the distance is outside of.

As another example, processor 132 can execute application instructions 146 to determine whether a turbidity and/or clarity (as indicated in physiological information 504a and/or 504b) of the captured scene information 506a is outside that specified by threshold ranges 512. As another example, processor 132 can execute application instructions 146 to determine whether the image saturation of (as indicated in physiological information 504a and/or 504b) of the captured scene information 506a is outside that specified by threshold ranges 512. In yet another example, processor 132 can execute application instructions 146 to determine whether the visibility of an aiming beam for the laser energy (as indicated in physiological information 504a and/or 504b) of the captured scene information 506a is outside that specified by threshold ranges 512.

Continuing to block 610 “generate a number of graphical elements from the physiological information and the determination of whether the physiological information is outside the threshold ranges based on the preferences” several graphical elements 516 can be generated from the physiological information 504a and 504b (including the captured scene information 506a and radiological image information 508a) and the threshold delta 514 based on the procedure preferences 502. In general, processor 132 can execute application instructions 146 to generate graphical elements 516 representative of physiological information 504a and 504b associated with each component in the OR suite (e.g., endoscope 204, fluidics unit 228, laser energy console 246, etc.) Further, the graphical elements 516 visually depict represented information using images, text, icons, colors, movement, or the like. With some embodiments, the graphical elements 516 can be like “alerts” or pop-up graphics.

For example, processor 132 can execute application instructions 146 to generate graphical elements 516 comprising an indication of the captured scene information 506a. As another example, processor 132 can execute application instructions 146 to generate graphical elements 516 comprising an indication of an ILP and/or flow rate. As another example, processor 132 can execute application instructions 146 to generate graphical elements 516 comprising an indication of a size of the stone 280 or a composition of the stone 280. As another example, processor 132 can execute application instructions 146 to generate graphical elements 516 comprising an indication of an intensity of the laser energy incidence of the stone 280, a distance between the distal end 260 and the stone 280, or the like.

In some embodiments, the information visually represented in graphical elements 516 is based on procedure preferences 502. For example, the information is based on the procedure type and equipment provisions, which is indicated in procedure preferences 502. As a further example, a first physician may prefer to pay attention to a first subset of physiological information 504a and 504b while another physician may prefer to pay attention to a second subset of physiological information 504a and 504b. In general, the first and second subsets may overlap, but this is not required.

Continuing to block 612 “generate, for each room of the OR suite, a composite display based on the graphical elements” composite displays 518 comprising multiple graphical elements 516 can be generated for each room or geographic location of the OR suite. For example, a composite displays 518 can be generated for each internal operating room display 102 or external operating room display 104. As outlined above, each composite displays 518 may comprise different combinations of graphical elements 516 depending upon which physical display (e.g., theater display 222, or the like) the composite displays 518 is to be displayed on.

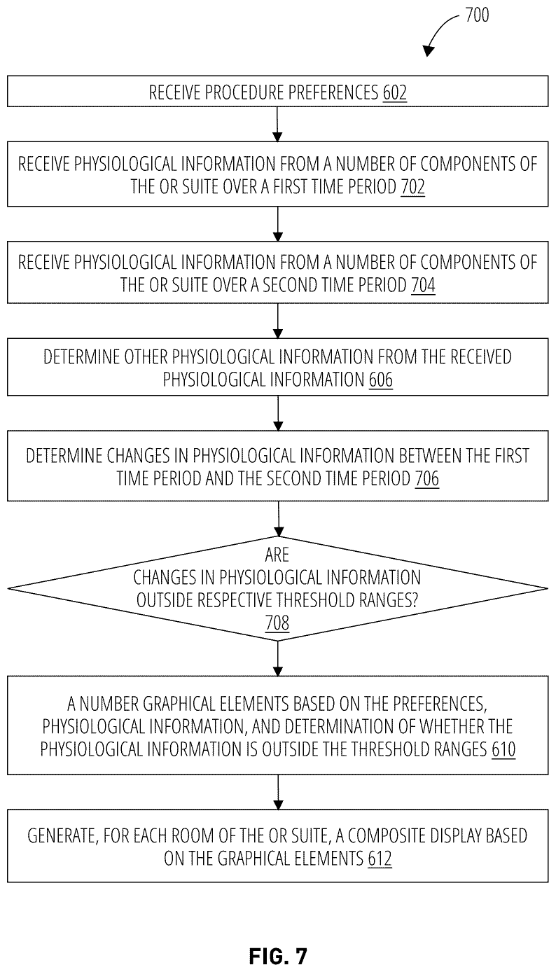

In some examples, graphical elements 516 can be generated based on a change in physiological information 504a and/or 504b. FIG. 7 illustrates logic flow 700, which can be carried out by centralized operating theater controller 500 to provide a centralized display of information. Logic flow 700 is much like logic flow 600 and where operations overlap, logical actions from logic flow 600 are reused in logic flow 700.

Logic flow 700, like logic flow 600, can begin with block 602. Continuing to block 702 “receive physiological information from a number of components of the OR suite over a first time period” and then to block 704 “receive physiological information from a number of components of the OR suite over a first time period” physiological data can be received from components of the OR environment 200 during a first time period (e.g., at block 702) and a second time period (e.g., at block 704). Blocks 702 and 704 of logic flow 700 are like block 604 of logic flow 600, except that blocks 702 and 704 receive physiological information 504a and 504c in respective first and second time periods. In some embodiments, the second time period is after the first time period. With some embodiments, the first and the second time periods can be snapshots in time separated by a time duration (e.g., 1 second(s), 0.5 s, 0.1 s, 0.01 s, 0.001 s, 0.0001 s, between 0.5 s and 1 s, or between 0.001 s and 0.1 s).

Logic flow 700 continues with block 606 from logic flow 600. However, as there is physiological information from two time periods (e.g., physiological information 504a and 504c), processor 132 can execute application instructions 146 to cause centralized operating theater controller 500 to derive and/or infer other physiological information 504b and 504d from physiological information 504a and 504c, respectively.

Continuing to block 706 “determine changes in physiological information between the first time period and the second time period” changes in physiological information from the first time period to the second time period can be determined. For example, processor 132 can execute application instructions 146 to determine changes in physiological information 520 based on the difference between physiological information 504a and 504c and physiological information 504b and 504d. As a specific example, processor 132 can execute application instructions 146 to determine a change in the size of stone 280 from the first time period (e.g., physiological information 504a and 504c) to the second time period (e.g., physiological information 504b and 504d). As another example, processor 132 can execute application instructions 146 to determine a change in the composition of the stone 280 from the first time period (e.g., physiological information 504a and 504c) to the second time period (e.g., physiological information 504b and 504d). As another example, processor 132 can execute application instructions 146 to determine a motion of the stone 280 between the first time period (e.g., physiological information 504a and 504c) and the second time period (e.g., physiological information 504b and 504d).

Continuing to decision block 708 “are changes in physiological information outside respective threshold ranges?” a determination of whether the changes in physiological information from the first time period to the second time period are outside respective threshold ranges is made. Decision block 708 of logic flow 700 can be like block 608 of logic flow 600 except that in logic flow 700, the determination is made with respect to changes in physiological information 520 and not the physiological information itself.

Logic flow 700 can continue with blocks 610 and 612 where graphical elements 516 and composite displays 518 can be generated. In some embodiments, centralized operating theater controller 500 can implement both logic flow 600 and logic flow 700 simultaneously.

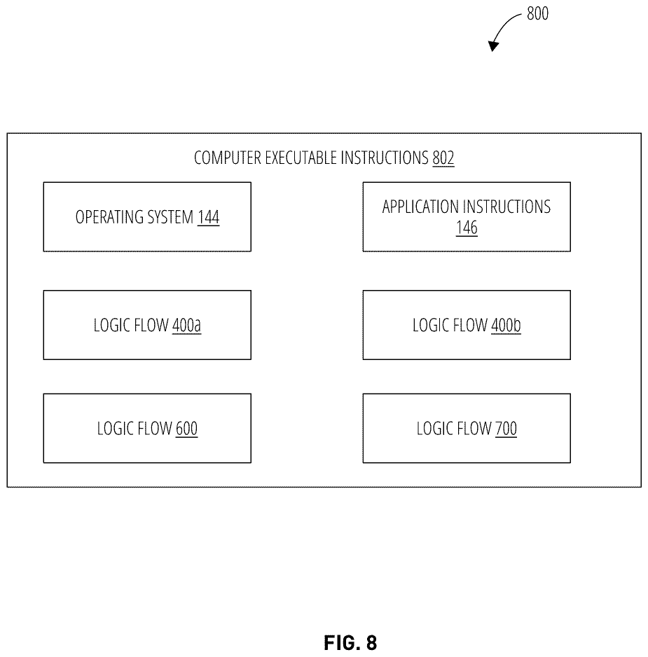

FIG. 8 illustrates computer-readable storage medium 800. Computer-readable storage medium 800 may comprise any non-transitory computer-readable storage medium or machine-readable storage medium, such as an optical, magnetic or semiconductor storage medium. In various embodiments, computer-readable storage medium 800 may comprise an article of manufacture. In some embodiments, computer-readable storage medium 800 may store computer executable instructions 802 with which circuitry (e.g., processor 132, or the like) can execute. For example, computer executable instructions 802 can include instructions to implement operations described with respect to operating system 144, application instructions 146, logic flow 400a, logic flow 400b, logic flow 600, and/or logic flow 700. Examples of computer-readable storage medium 800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 802 may include any suitable type of code, such as source code, compiled code, interpreted code, executable code, static code, dynamic code, object-oriented code, visual code, and the like.

Terms used herein should be accorded their ordinary meaning in the relevant arts, or the meaning indicated by their use in context, unless an express definition is provided, in which case the definition provided herein controls. Additionally, references to “one embodiment” or “an embodiment” do not necessarily refer to the same embodiment. Further, embodiments can be combined where combination does not conflict with the context provided. Words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application.

When the claims use the word “or” in reference to a list of two or more items, that word covers all the following interpretations of the word: any of the items in the list, all the items in the list, and any combination of the items in the list, unless expressly limited to one or the other. When the claims use the word “and/or” in reference to a list of two or more items, that word covers any combination of the listed items. For example, where a claim recites “item 1, item 2, and/or item 3,” the claim means item 1 alone, item 2 alone, item 3 alone, items 1 and 2, items 1 and 3, items 2 and 3, or items 1, 2, and 3.

What is claimed is:

1. A urological operating room suite comprising:
an operating theater;
a plurality of therapy consoles provisioned in the operating theater, each of the plurality of therapy consoles comprising processing circuitry, wherein the plurality of therapy consoles are configured to be utilized simultaneously to perform a urological procedure in the operating room (OR) suite;
an operating theater display provisioned in the operating theater; and
a centralized operating theater controller, the centralized operating theater controller comprising a processor and a memory storage device coupled to the processor, the memory storage device comprising instructions that when executed by the processor cause the centralized operating theater controller to:
receive, from the processing circuitry of at least one of the plurality of therapy consoles, an indication of physiological information related to the urological procedure;
generate a plurality of graphical elements comprising visual indications of the physiological information;
generate a composite display based on the plurality of graphical elements; and
send control signals to the operating theater display to cause the operating theater display to display the composite display.

2. The urological operating room suite of claim 1, the instructions when executed by the processor, further cause the centralized operating theater controller to:
determine other physiological information from the received physiological information,
wherein the plurality of graphical elements further comprises visual indications of the other physiological information.

3. The urological operating room suite of claim 2, wherein the plurality of therapy consoles comprises at least a fluidics unit, a laser energy console, and an endoscope, and wherein the received physiological information comprises one or more of a pressure, a fluid flow, an energy, or a power, and wherein the determined other physiological information comprises a turbidity, a clarity, a temperature, a procedure state, or a target tissue state.

4. The urological operating room suite of claim 1, the instructions when executed by the processor, further cause the centralized operating theater controller to:
receive, from the processing circuitry of at least one of the plurality of therapy consoles, an indication of captured scene information,
wherein the plurality of graphical elements further comprises visual indications of the captured scene information.

5. The urological operating room suite of claim 1, further comprising an observation room and an observation room display provisioned in the observation room, wherein the composite display is a first composite display and wherein the instructions when executed by the processor, further cause the centralized operating theater controller to:
generate a second composite display based on one or more first ones of the plurality of graphical elements; and
send control signals to the observation room display to cause the observation room display to display the second composite display,
wherein the first composite display is generated based on one or more second ones of the plurality of graphical elements.

6. The urological operating room suite of claim 5, wherein the one or more first ones of the plurality of graphical elements are different than the one or more second ones of the plurality of graphical elements and wherein the first composite display is different than the second composite display.

7. The urological operating room suite of claim 1, further comprising one or more equipment controls, wherein the instructions when executed by the processor, further cause the centralized operating theater controller to:
receive, from at least one of the one or more equipment controls, at least one control signal, the at least one control signal comprising indications to cause a one of the plurality of therapy consoles to change a parameter or maintain a parameter; and
send, to the one of the plurality of therapy consoles, the at least one control signal.

8. The urological operating room suite of claim 7, wherein the instructions when executed by the processor, further cause the centralized operating theater controller to process the at least one control signal, wherein the processed at least one control signal is sent to the one of the plurality of therapy consoles.

9. The urological operating room suite of claim 8, wherein the instructions when executed by the processor, further cause the centralized operating theater controller to send the processed at least one control signal to another one of the plurality of therapy consoles.

10. The urological operating room suite of claim 9, wherein the instructions when executed by the processor, further cause the centralized operating theater controller to:
receive, from the one of the plurality of therapy consoles, a feedback signal;
process the received feedback signal; and
send, to the at least one of the one or more equipment controls, the processed feedback signal, wherein the processed feedback signal causes the at least one of the one or more equipment controls to provide feedback to a user.

11. The urological operating room suite of claim 10, wherein the one or more equipment controls are provisioned in the operating theater.

12. The urological operating room suite of claim 1, wherein the instructions when executed by the processor, further cause the centralized operating theater controller to:
generate configuration signals for the plurality of therapy consoles based in part on procedure preferences for the urological procedure;
send, to the plurality of therapy consoles, the configuration signals.

13. A method for a centralized operating theater controller, comprising:
receiving, from processing circuitry of at least one of a plurality of therapy consoles, an indication of physiological information related to a urological procedure;
generating a plurality of graphical elements comprising visual indications of the physiological information;
generating a composite display based on the plurality of graphical elements; and
sending control signals to an operating theater display to cause the operating theater display to display the composite display,
wherein the operating theater display is provisioned in an operating theater of a urological operating room (OR) suite, and
wherein the plurality of therapy consoles are provisioned in the operating theater and configured to be utilized simultaneously to perform the urological procedure in the urological OR suite.

14. The method of claim 13, further comprising:
determining other physiological information from the received physiological information,
wherein the plurality of graphical elements further comprises visual indications of the other physiological information.

15 30. The method of claim 14, wherein the plurality of therapy consoles comprises at least a fluidics unit, a laser energy console, and an endoscope, and wherein the received physiological information comprises one or more of a pressure, a fluid flow, an energy, or a power, and wherein the determined other physiological information comprises a turbidity, a clarity, a temperature, a procedure state, or a target tissue state.

16. The method of claim 13, further comprising:
receiving, from the processing circuitry of at least one of the plurality of therapy consoles, an indication of captured scene information,
wherein the plurality of graphical elements further comprises visual indications of the captured scene information.

17. The method of claim 13, wherein the composite display is a first composite display, the method further comprising:
generating a second composite display based on one or more first ones of the plurality of graphical elements; and
sending control signals to an observation room display to cause the observation room display to display the second composite display,
wherein the first composite display is generated based on one or more second ones of the plurality of graphical elements, and
wherein the observation room display is provisioned in an observation room of the urological OR suite.

18. At least one machine readable storage device, comprising a plurality of instructions that in response to being executed by a processor of a centralized operating theater controller cause the centralized operating theater controller to:
receive, from processing circuitry of at least one of a plurality of therapy consoles, an indication of physiological information related to a urological procedure;
generate a plurality of graphical elements comprising visual indications of the physiological information;
generate a composite display based on the plurality of graphical elements; and
send control signals to an operating theater display to cause the operating theater display to display the composite display,
wherein the operating theater display is provisioned in an operating theater of a urological operating room (OR) suite, and
wherein the plurality of therapy consoles are provisioned in the operating theater and configured to be utilized simultaneously to perform the urological procedure in the urological OR suite.

19. The at least one machine readable storage device of claim 18, wherein the instructions when executed by the processor, further cause the centralized operating theater controller to:
receive, from at least one of one or more equipment controls, at least one control signal, the at least one control signal comprising indications to cause a one of the plurality of therapy consoles to change a parameter or maintain a parameter; and
send, to the one of the plurality of therapy consoles, the at least one control signal.

20. The at least one machine readable storage device of claim 19, wherein the instructions when executed by the processor, further cause the centralized operating theater controller to:
receive, from the one of the plurality of therapy consoles, a feedback signal;
process the received feedback signal; and
send, to the at least one of the one or more equipment controls, the processed feedback signal, wherein the processed feedback signal causes the at least one of the one or more equipment controls to provide feedback to a user.
